(19) United States
(12) Patent Application Publication
(10) Pub. No.: US 2020/0390766 A1
(43) Pub. Date: Dec. 17, 2020

(54) PHARMACEUTICAL COMPOSITION

(71) Applicant: Kowa Company, Ltd., Nagoya (JP)

(72) Inventors: Kentaro Mikami, Tokyo (JP); Rie Ishida, Tokyo (JP); Takashi Nakagawa, Tokyo (JP)

(21) Appl. No.: 16/899,638

(22) Filed: Jun. 12, 2020

Related U.S. Application Data
(60) Provisional application No. 62/860,971, filed on Jun. 13, 2019.

(51) Int. Cl. A61K 31/504; A61P 11/00

(52) U.S. Cl. CPC A61K 31/504; A61P 11/00

(57) ABSTRACT

[Problem to be solved]

The present invention addresses the problem of providing a pharmaceutical composition for the prevention and/or treatment of diseases related to pulmonary fibrosis.

[Solution]

Provided is a pharmaceutical composition for the prevention and/or treatment of diseases involving pulmonary fibrosis, in which the active ingredient is 3-[2-(5-{[1-(5-ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one, or a salt thereof, or a solvate of these.

### TECHNICAL FIELD

The present invention relates to prevention and/or treatment of pulmonary fibrosis related disorders.

### BACKGROUND ART

Pulmonary fibrosis is caused by inflammation or damage of alveolar walls. As the pulmonary fibrosis progresses, gas exchange between alveoli and capillaries cannot be performed smoothly, so that symptoms such as shortness of breath and malaise during daily activities are exhibited.

As the pulmonary fibrosis related disorders, autoimmune diseases such as rheumatoid arthritis and polydermatomyositis, pneumoconiosis caused by inhalation of dust, drug-induced pneumonia caused by side effects of drugs, special infectious diseases and the like are known, and the lung fibrogenesis progresses for various reasons. Meanwhile, among the pulmonary fibrosis related disorders, there are diseases whose cause cannot be identified, which are classified as idiopathic interstitial pneumonia.

Idiopathic interstitial pneumonia is, as a clinicopathological entity, classified into idiopathic pulmonary fibrosis, non-specific interstitial pneumonia, cryptogenic organizing pneumonia, desquamative interstitial pneumonia, lymphocytic interstitial pneumonia, acute interstitial pneumonia, respiratory bronchiolitis-interstitial lung disease, and the like. Among these diseases included in idiopathic interstitial pneumonia, idiopathic pulmonary fibrosis is the most common, which is a very serious disease with an average survival of 3 to 5 years after diagnosis (Non-Patent Document 1). In patients with idiopathic pulmonary fibrosis, fibrosis of the lungs progresses gradually, but within a few days to a month, disease condition can rapidly progress to an acute exacerbation that result in respiratory failure, and in some cases, death.

In the study of idiopathic pulmonary fibrosis, model mice that have induced pulmonary fibrosis by exposure to bleomycin are widely used (Non-Patent Document 2). In pathological findings, the model mice have characteristics similar to those of idiopathic pulmonary fibrosis in clinical practice, such as showing an increase in inflammatory cells and an increase in the amount of hydroxyproline, a collagen component constituting an extracellular matrix, which is an indicator of fibrosis, thus is also used for research and development of therapeutic agents (Patent Document 1).

For idiopathic pulmonary fibrosis, there is currently no treatment to cure the disease. Therefore, in the treatment of idiopathic pulmonary fibrosis, drug administration is performed to suppress the progression of disease condition. As medicines, only pirfenidone and nintedanib are approved in Japan. However, effects of either drug only slow the progression of disease. Moreover, there is a group of patients for which no significant effect is observed after administration, and when side effects are confirmed, it may be required to stop taking the drug, thus sufficient treatment has not yet been provided. In addition, although a clinical trial of losartan adapted for treatment of hypertension and the like for idiopathic pulmonary fibrosis has been carried out in the United States, it has not yet been put to practical use. Under these circumstances, a new therapeutic agent is still desired.

3-[2-(5-{[1-(5-Ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one is a compound known to have angiotensin II receptor antagonistic activity (Patent Document 2). This compound is expected, for example, as prophylactic and/or therapeutic agents for diseases such as hypertension, heart disease, angina, cerebrovascular disorder, cerebral circulatory disorder, ischemic peripheral circulatory disorder, renal disease, arteriosclerosis, type 2 diabetes, diabetic complications, insulin resistance syndrome, metabolic syndrome, and hyperinsulinemia. However, there is no report on effect of this compound on pulmonary fibrosis related disorders.

### CITATION LIST

### Patent Document

[Patent Document 1] WO 2010/137160 A1
[Patent Document 2] WO 2012/124311 A1

### Non-Patent Document

[Non-Patent Document 1] Meyer K, et al.; Therapeutics and Clinical Risk Management 2017 Apr. 3; 13: 427-437
[Non-Patent Document 2] Moeller A, et al.; Int. J. Biochem. Cell Biol. 2008; 40(3): 362-382

### SUMMARY OF THE INVENTION

### Technical Problem

An object of the present invention is to provide a new pharmaceutical useful for preventing and/or treating pulmonary fibrosis related disorders.

### Means for Solving the Problem

In order to achieve the above object, the present inventors have conducted intensive studies, and found that, surprisingly, by administering the compound disclosed as Example 40 in Patent Document 2, 3-[2-(5-{[1-(5-ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one (hereinafter sometimes referred to as “compound A”) to a disease model animal, the amount of hydroxyproline in the lungs is reduced, and an increase in lung weight is suppressed. From these effects, the present inventors have found that compound A is useful for treating pulmonary fibrosis related disorders, and completed the present invention.

That is, the present invention provides a pharmaceutical composition comprising 3-[2-(5-{[1-(5-ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one or a salt, a solvate or a solvate of the salt thereof for preventing and/or treating a pulmonary fibrosis related disorder.

### Advantageous Effects of the Invention

The present invention provides a new pharmaceutical composition useful for preventing and/or treating pulmonary fibrosis related disorders. According to the present invention, a means for preventing diseases by suppressing fibrosis of the lungs of patients to whom a pharmaceutical composition has been administered is provided, and a new treatment option is provided for patients with a pulmonary fibrosis related disorder in which an effect cannot be sufficiently recognized with current therapeutic agents.

### BRIEF DESCRIPTION OF DRAWINGS

FIG. 1 illustrates measurement results of lung weight in Example 1. In the figure, * indicates a significant difference (Dunnett test) of p<0.05 as compared to a normal group, and *** indicates a significant difference (t test) of p<0.001 as compared to the normal group, respectively. In addition, † indicates that a significant difference (Dunnett test) of p<0.05 as compared to a control group.

FIG. 2 illustrates measurement results of the amount of hydroxyproline in Example 1. In the figure, *** indicates a significant difference (t test) of p<0.001 as compared to the normal group. In addition, † indicates a significant difference of p<0.05 as compared to the control group, and †† indicates a significant difference of p<0.01 as compared to the control group (both Dunnett test).

FIG. 3 illustrates measurement results of the amount of hydroxyproline in Example 2. In the figure, *** indicates a significant difference (t test) of p<0.001 as compared to the normal group. In addition, † indicates a significant difference (Dunnett test) of p<0.05 as compared to the control group.

### DESCRIPTION OF EMBODIMENTS

3-[2-(5-{[1-(5-Ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one used in the present invention has a structure shown in the following formula (1):

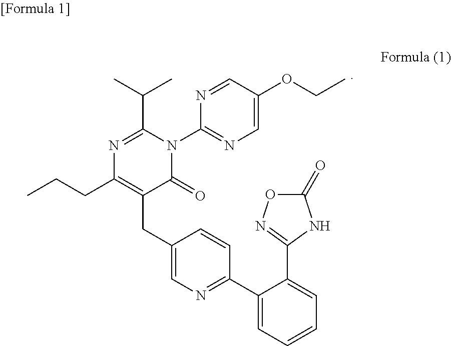 [SMILES: CCCc1nc(C(C)C)n(-c2ncc(OCC)cn2)c(=O)c1Cc1ccc(-c2ccccc2-c2noc(=O)[nH]2)nc1]

The compound can be produced, for example, according to the method described in WO 2012/124311 or the like. Further, the compound can be formed into a preparation according to the method described in the Document or the like.

Moreover, in practicing the present invention, a salt or solvate of compound A can be used instead of compound A. The salt of compound A is not particularly limited as long as it is a pharmaceutically acceptable salt. When the compound is treated as an acidic compound, examples of the salt include salts with alkali metal or alkaline earth metal such as sodium, potassium, magnesium or calcium; salts with an organic base such as trimethylamine, triethylamine, pyridine, picoline, N-methylpyrrolidine, N-methylpiperidine or N-methylmorpholine; and the like. When the compound is treated as a basic compound, examples of the salt include mineral acid addition salts, such as hydrochlorides, hydrobromides, hydroiodides, sulfates, nitrates and phosphates; organic acid addition salts, such as benzoates, methane sulfonates, ethane sulfonates, benzene sulfonates, p-toluene sulfonates, maleates, fumarates, tartrates, citrates and acetates; and the like. Examples of the solvate of compound A or the salt of compound A include hydrates and solvates with ethanol and the like, but are not limited thereto.

The “pulmonary fibrosis related disorders” which can be prevented and/or treated by a pharmaceutical composition comprising compound A, a salt, a solvate or the solvate of the salt thereof used in the present invention are not limited to the diseases in which fibrosis of the lungs is found, but encompass inflammation or damage to the alveolar walls that cause fibrotic lung.

Examples of diseases included in the “pulmonary fibrosis related disorder” include idiopathic interstitial pneumonia (including idiopathic pulmonary fibrosis, non-specific interstitial pneumonia, cryptogenic organizing pneumonia, desquamative interstitial pneumonia, lymphocytic interstitial pneumonia, acute interstitial pneumonia, and respiratory bronchiolitis-associated interstitial lung disease), diffuse interstitial pneumonia, post-inflammatory pulmonary fibrosis, usual interstitial pneumonia, pulmonary fibrosis, diffuse alveolar damage, autoimmune disease (rheumatoid arthritis, polydermatomyositis, and the like) and interstitial pneumonia caused by the autoimmune disease, pneumoconiosis, chronic hypersensitivity pneumonia and interstitial pneumonia caused by the chronic hypersensitivity pneumonia, drug-induced pneumonia (pneumonia induced by drugs such as bleomycin, gefitinib, and Shosaikoto) and interstitial pneumonia caused by the drug-induced pneumonia, viral infectious disease (viral infectious disease caused by cytomegalovirus, influenza virus, and the like) and interstitial pneumonia caused by the viral infectious disease, radiation pneumonitis and interstitial pneumonia caused by the radiation pneumonitis, sarcoidosis and interstitial pneumonia caused by the sarcoidosis, pulmonary involvements associated with collagen vascular disease and interstitial pneumonia caused by the disease, and systemic scleroderma and interstitial pneumonia caused by the systemic scleroderma.

In another embodiment of the present invention, a pharmaceutical composition comprising compound A, a salt, a solvate or a solvate of the salt thereof can be used for suppressing an increase in the amount of hydroxyproline in the lungs of a patient.

In another embodiment of the present invention, a pharmaceutical composition comprising compound A, a salt, a solvate or a solvate of the salt thereof can be used for suppressing an increase in lung weight of a patient.

In another embodiment of the present invention, a pharmaceutical composition comprising compound A, a salt, a solvate or a solvate of the salt thereof can be used for maintaining respiratory function of a patient (including suppressing a decrease in respiratory function).

Examples of index of respiratory function include vital capacities (including vital capacity (VC), % VC, forced vital capacity (FVC), % FVC, and the like), gas exchange functions (including arterial oxygen tension (PaO₂), arterial oxygen saturation (SpO₂), diffusion capacity of the lungs for carbon monoxide (DLCO), and the like) and the like, but are not limited thereto. In addition, measurement of vital capacities and gas exchange functions can be performed by generally known methods.

The measurement results of respiratory function may be used to evaluate progression of pulmonary fibrosis. For example, in the Ministry of Health, Labor and Welfare, the severity of idiopathic pulmonary fibrosis is determined based on PaO₂ at rest and SpO₂ during walking for 6 minutes of a patient, as shown in Table 1. In this severity classification, as the disease becomes more severe, the numerical value of the classification increases, and the severity classification IV is the most severe.

TABLE 1

| Severity | PaO₂ | SpO₂ |
|---|---|---|
| classification | at rest | during 6-min walk |
| I | ≥80 Torr | |
| II | ≥70 Torr | Classified as classification III |
| | <80 Torr | when less than 90% |
| III | ≥60 Torr | Classified as classification IV |
| | <70 Torr | when less than 90% |
| | | (unnecessary in cases with danger) |
| IV | <60 Torr | unnecessary |

In addition, an another scale has been proposed for the severity of idiopathic pulmonary fibrosis, called the “GAP score (GAP index)” (Ann. Intern. Med. 2012; 156: 684-691). In this scale, an item to which a patient corresponds is selected from the evaluation items, point addition is performed for each item, and the patient is classified based on the total points. Tables 2 and 3 show the evaluation items and the classification based on the total points, respectively. In this severity classification, as the disease becomes more severe, the numerical value of the classification increases, and the severity classification III is the most severe.

TABLE 2

| | Patient | Points |
|---|---|---|
| Gender | Female | 0 |
| | Male | 1 |
| Age, y | ≤60 | 0 |
| | 61-65 | 1 |
| | >65 | 2 |
| FVC, % | >75 | 0 |
| | 50-75 | 1 |
| | <50 | 2 |
| DLCO, % | >55 | 0 |
| | 36-55 | 1 |
| | <35 | 2 |
| | Cannot perform | 3 |

In the table, % FVC is the measurement result of forced vital capacity expressed as a percentage of the predicted value. Also, % DLCO is the measurement result of diffusion capacity of the lungs for carbon monoxide expressed as a percentage of the predicted value.

TABLE 3

| Severity | |
|---|---|
| classification | |
| (Stage) | Points |
| I | 0-3 |
| II | 4-5 |
| III | 6-8 |

In one embodiment of the present invention, examples of a dosage form of a pharmaceutical composition or an administration form thereof, comprising compound A, a salt, a solvate or a solvate of the salt thereof, include oral administration by tablets, capsules, granules, powders, syrups and the like, and parenteral administration by intravenous injection, intramuscular injection, suppository, inhalant, transdermal absorbent, eye drop, nasal drop and the like. Moreover, in order to prepare pharmaceutical preparations of such various dosage forms, compound A, a salt, a solvate or a solvate of the salt thereof can be prepared alone or as a pharmaceutical composition by appropriately combining other pharmaceutically acceptable carriers, that is, an excipient, a binding agent, a bulking agent, a disintegrant, a surfactant, a lubricant, a dispersant, a buffer, a preservative, a flavor, a fragrance, a coating agent, a diluent, and the like.

Examples of the pulmonary fibrosis related disorder to be prevented and/or treated by the pharmaceutical composition comprising compound A, a salt, a solvate or a solvate of the salt thereof include idiopathic interstitial pneumonia (including idiopathic pulmonary fibrosis, non-specific interstitial pneumonia, cryptogenic organizing pneumonia, desquamative interstitial pneumonia, lymphocytic interstitial pneumonia, acute interstitial pneumonia, and respiratory bronchiolitis-associated interstitial lung disease), diffuse interstitial pneumonia, post-inflammatory pulmonary fibrosis, usual interstitial pneumonia, pulmonary fibrosis, diffuse alveolar damage, autoimmune disease (rheumatoid arthritis, polydermatomyositis, and the like) and interstitial pneumonia caused by the autoimmune disease, pneumoconiosis, chronic hypersensitivity pneumonia and interstitial pneumonia caused by the chronic hypersensitivity pneumonia, drug-induced pneumonia (pneumonia induced by drugs such as bleomycin, gefitinib, and Shosaikoto) and interstitial pneumonia caused by the drug-induced pneumonia, viral infectious disease (viral infectious disease caused by cytomegalovirus, influenza virus, and the like) and interstitial pneumonia caused by the viral infectious disease, radiation pneumonitis and interstitial pneumonia caused by the radiation pneumonitis, sarcoidosis and interstitial pneumonia caused by the sarcoidosis, pulmonary involvements associated with collagen vascular disease and interstitial pneumonia caused by the disease, and systemic scleroderma and interstitial pneumonia caused by the systemic scleroderma.

Among them, preferred are usual interstitial pneumonia, interstitial pneumonia caused by autoimmune disease, interstitial pneumonia caused by drug-induced pneumonia, interstitial pneumonia caused by viral infectious disease, interstitial pneumonia caused by radiation pneumonitis, interstitial pneumonia caused by sarcoidosis, interstitial pneumonia caused by pulmonary involvements associated with collagen vascular disease, interstitial pneumonia caused by systemic scleroderma, and idiopathic interstitial pneumonia (including idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia), and idiopathic pulmonary fibrosis is a particularly preferred target disease.

One embodiment of the present invention includes the following [1] to [6].

[1] A pharmaceutical composition comprising 3-[2-(5-{[1-(5-ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one, a salt, a solvate or a solvate of the salt thereof for preventing and/or treating a pulmonary fibrosis related disorder.

[2] The pharmaceutical composition according to above [1], wherein the pulmonary fibrosis related disorder is a disease selected from the group consisting of idiopathic interstitial pneumonia, diffuse interstitial pneumonia, post-inflammatory pulmonary fibrosis, usual interstitial pneumonia, pulmonary fibrosis, diffuse alveolar damage, autoimmune disease and interstitial pneumonia caused by the autoimmune disease, pneumoconiosis, chronic hypersensitivity pneumonia and interstitial pneumonia caused by the chronic hypersensitivity pneumonia, drug-induced pneumonia and interstitial pneumonia caused by the drug-induced pneumonia, viral infectious disease and interstitial pneumonia caused by the viral infectious disease, radiation pneumonitis and interstitial pneumonia caused by the radiation pneumonitis, sarcoidosis and interstitial pneumonia caused by the sarcoidosis, pulmonary involvements associated with collagen vascular disease and interstitial pneumonia caused by the disease, and systemic scleroderma and interstitial pneumonia caused by the systemic scleroderma.

[3] The pharmaceutical composition according to above [1], wherein the pulmonary fibrosis related disorder is a disease selected from the group consisting of usual interstitial pneumonia, interstitial pneumonia caused by autoimmune disease, interstitial pneumonia caused by drug-induced pneumonia, interstitial pneumonia caused by viral infectious disease, interstitial pneumonia caused by radiation pneumonitis, interstitial pneumonia caused by sarcoidosis, interstitial pneumonia caused by pulmonary involvements associated with collagen vascular disease, interstitial pneumonia caused by systemic scleroderma, and idiopathic interstitial pneumonia.

[4] The pharmaceutical composition according to above [2] or [3], wherein the idiopathic interstitial pneumonia is a disease selected from the group consisting of idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia.

[5] The pharmaceutical composition according to above [1], wherein the pulmonary fibrosis related disorder is a disease selected from the group consisting of idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia.

[6] The pharmaceutical composition according to above [1], wherein the pulmonary fibrosis related disorder is idiopathic pulmonary fibrosis.

Further, one embodiment of the present invention includes the following [a] to [c].

[a] A method for preventing and/or treating a pulmonary fibrosis related disorder, comprising administering compound A, a salt, a solvate or a solvate of the salt thereof to a subject.

[b] Compound A, a salt, a solvate or a solvate of the salt thereof for use in prevention and/or treatment of a pulmonary fibrosis related disorder.

[c] Use or application of compound A, a salt, a solvate or a solvate of the thereof in the production of a pharmaceutical composition for prevention and/or treatment of a pulmonary fibrosis related disorder.

In [a] to [c], as the pulmonary fibrosis related disorder, idiopathic interstitial pneumonia (including idiopathic pulmonary fibrosis, non-specific interstitial pneumonia, cryptogenic organizing pneumonia, desquamative interstitial pneumonia, lymphocytic interstitial pneumonia, acute interstitial pneumonia, and respiratory bronchiolitis-associated interstitial lung disease), diffuse interstitial pneumonia, post-inflammatory pulmonary fibrosis, usual interstitial pneumonia, pulmonary fibrosis, diffuse alveolar damage, autoimmune disease (rheumatoid arthritis, polydermatomyositis, and the like) and interstitial pneumonia caused by the autoimmune disease, pneumoconiosis, chronic hypersensitivity pneumonia and interstitial pneumonia caused by the chronic hypersensitivity pneumonia, drug-induced pneumonia (pneumonia induced by drugs such as bleomycin, gefitinib, and Shosaikoto) and interstitial pneumonia caused by the drug-induced pneumonia, viral infectious disease (viral infectious disease caused by cytomegalovirus, influenza virus, and the like) and interstitial pneumonia caused by the viral infectious disease, radiation pneumonitis and interstitial pneumonia caused by the radiation pneumonitis, sarcoidosis and interstitial pneumonia caused by the sarcoidosis, pulmonary involvements associated with collagen vascular disease and interstitial pneumonia caused by the disease, and systemic scleroderma and interstitial pneumonia caused by the systemic scleroderma are preferred, usual interstitial pneumonia, interstitial pneumonia caused by autoimmune disease, interstitial pneumonia caused by drug-induced pneumonia, interstitial pneumonia caused by viral infectious disease, interstitial pneumonia caused by radiation pneumonitis, interstitial pneumonia caused by sarcoidosis, interstitial pneumonia caused by pulmonary involvements associated with collagen vascular disease, interstitial pneumonia caused by systemic scleroderma, and idiopathic interstitial pneumonia (including idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia) are further preferred, and idiopathic pulmonary fibrosis is particularly preferred.

One embodiment of the present invention further includes the following [d] to [g].

[d] A pharmaceutical composition comprising compound A, a salt, a solvate or a solvate of the salt thereof for suppressing progression of idiopathic pulmonary fibrosis.

[e] A method for suppressing progression of idiopathic pulmonary fibrosis, comprising administering compound A, a salt, a solvate or a solvate of the salt thereof to a subject with idiopathic pulmonary fibrosis.

[f] Compound A, a salt, a solvate or a solvate of the salt thereof for use in suppressing progression of idiopathic pulmonary fibrosis.

[g] Use of compound A, a salt, a solvate or a solvate of the salt thereof for the production of a pharmaceutical for suppressing progression of idiopathic pulmonary fibrosis.

As an index of severity of idiopathic pulmonary fibrosis in [d] to [g], for example, one or both of the severity classification of idiopathic pulmonary fibrosis and the GAP score specified by the Ministry of Health, Labor and Welfare can be used.

In one embodiment of the present invention, the dose of the pharmaceutical composition, the number of administrations per day, and the administration form can be appropriately determined according to the patient's body weight, age, sex, symptoms, and the like.

Hereinafter, the present invention will be described in more detail with reference to Examples, but these Examples do not limit the present invention.

### EXAMPLES

### Example 1: Investigation of Preventive Effects on Pulmonary Fibrosis

[Sample Solution Preparation]

Compound A was dissolved in a 0.5% aqueous solution of methylcellulose (methylcellulose: Shin-Etsu Chemical Co., Ltd., distilled water: Otsuka Pharmaceutical Factory, Inc.) to prepare a sample solution. Further, losartan (Tokyo Chemical Industry Co., Ltd.) was dissolved in a 0.5% aqueous solution of methylcellulose to prepare a sample solution. Furthermore, pirfenidone (Ark Pharma Inc.) was dissolved in a 0.5% aqueous solution of methylcellulose to prepare a sample solution.

[Preparation of Model Animal]

6-week-old male ICR mice (Japan SLC, Inc.) were exposed to bleomycin (Nippon Kayaku Co., Ltd.) (1.5 mg/kg) dissolved in physiological saline in the trachea to prepare pulmonary fibrosis model mice.

[Test Method]

Investigation was conducted in mice not exposed to bleomycin (exposed to physiological saline in the trachea) in the normal group (6 mice), and pulmonary fibrosis model mice exposed to bleomycin in the trachea divided into 4 groups (control, compound A 50 mg/kg/day, losartan 50 mg/kg/day, and pirfenidone 400 mg/kg/day; 10 mice in each group). From the day after exposure to bleomycin, a 0.5% aqueous solution of methylcellulose was orally administered (administered twice a day) to the normal group and the control group, the sample solution of compound A prepared above was orally administered (administered once a day) to the compound A administration group, the sample solution of losartan prepared above was orally administered (administered once a day) to the losartan administration group, and the sample solution of pirfenidone prepared above was orally administered (administered twice a day) to the pirfenidone administration group, respectively. Twenty-one days after the bleomycin exposure, the mice were anesthetized and whole lung tissues were extracted.

Lung Weight

[Measurement Method]

After washing the lung tissue extracted from the mouse with physiological saline, water was removed with a filter paper, and the wet weight was measured.

[Measurement Result]

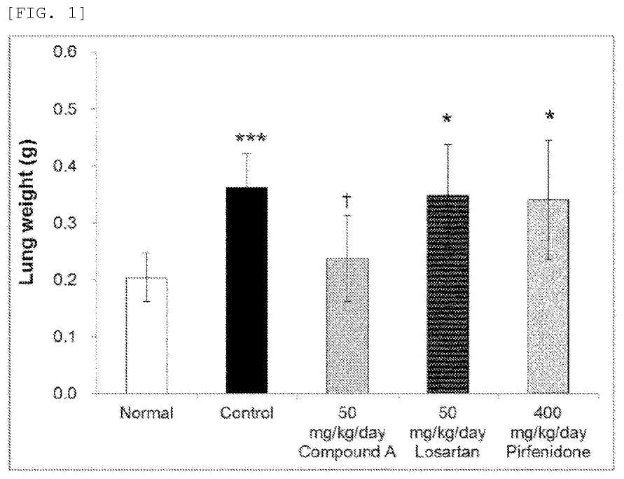

FIG. 1 shows the lung weight (g) of each group. In the figure, * indicates a significant difference (Dunnett test) of p<0.05 as compared to the normal group, and *** indicates a significant difference (t test) of p<0.001 as compared to the normal group, respectively. In addition, † indicates a significant difference (Dunnett test) of p<0.05 as compared to the control group. As is clear from FIG. 1, by intratracheal exposure to bleomycin, a significant increase in lung weight was observed in the control group, the losartan administration group and the pirfenidone administration group, but no significant increase in lung weight was observed in the compound A administration group. In addition, a significant decrease in lung weight was observed in the compound A administration group as compared with the control group. From these results, a suppression effect of compound A on the increase in lung weight was observed in pulmonary fibrosis induction by intratracheal exposure to bleomycin.

Amount of Hydroxyproline

[Measurement Method]

The lung tissue extracted from the mouse was homogenized in a PBS solution, then mixed with an equal volume of 12N hydrochloric acid in a tube, and incubated at 110° C. for 20 to 24 hours. After the incubation, the solution was volatilized by opening the tube and continuing the incubation. Thereafter, the resulting mixture was suspended in a citrate acetate buffer (5% citric acid, 1.2% glacial acetic acid, 7.25% sodium acetate, 3.4% sodium hydroxide, pH 6.0), and the solution filtered with a 0.45 μm diameter centrifugal filter was used as a sample. 10 μL of the sample was added with 100 μL of chloramine T solution (1.4% chloramine T, 10% n-propanol, 80% citrate acetate buffer) and incubated at room temperature for 20 minutes, then added with 100 μL of Ehrlich's reagent (1 M p-dimethylamino benzaldehyde, 20% perchloric acid, 50% n-propanol) and incubated at 65° C. for 15 minutes, and the absorbance at 550 nm was measured to quantify the amount of hydroxyproline.

[Measurement Result]

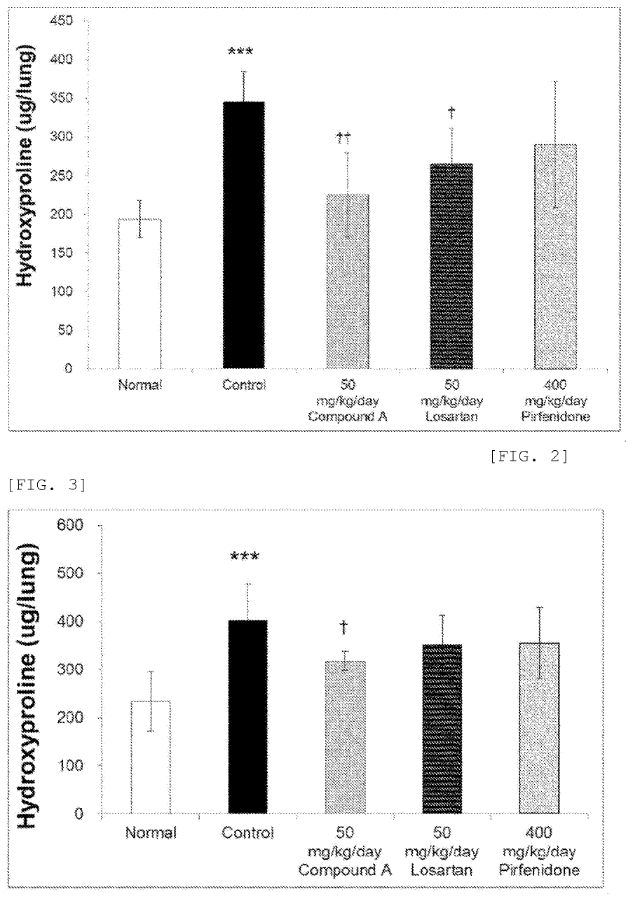

FIG. 2 shows the amount (μg) of hydroxyproline in the lungs of each group. In the figure, *** indicates a significant difference (t test) of p<0.001 as compared to the normal group. In addition, † indicates a significant difference of p<0.05 as compared to the control group, and †† indicates a significant difference of p<0.01 as compared to the control group (both Dunnett test). As is clear from FIG. 2, a significant increase in hydroxyproline in the lung was observed in the control group by intratracheal exposure to bleomycin. On the other hand, a significant decrease in hydroxyproline in the lung was observed in the compound A administration group and the losartan administration group as compared to the control group, and the compound A administration group showed larger decrease than the losartan administration group. A suppression effect of compound A on hydroxyproline accumulation in the lung was observed in pulmonary fibrosis induction by intratracheal exposure to bleomycin.

### Example 2: Investigation of Therapeutic Effects on Pulmonary Fibrosis

[Sample Solution Preparation]

Compound A was dissolved in a 0.5% aqueous solution of methylcellulose (methylcellulose: Shin-Etsu Chemical Co., Ltd., distilled water: Otsuka Pharmaceutical Factory, Inc.) to prepare a sample solution. Further, losartan (Tokyo Chemical Industry Co., Ltd.) was dissolved in a 0.5% aqueous solution of methylcellulose to prepare a sample solution. Furthermore, pirfenidone (Ark Pharma Inc.) was dissolved in a 0.5% aqueous solution of carboxymethyl cellulose (carboxymethyl cellulose: Maruishi Pharmaceutical Co., Ltd.) to prepare a sample solution.

[Preparation of Model Animal]

Model mice were prepared in the same manner as in Example 1.

[Test Method]

Investigation was conducted in mice not exposed to bleomycin (exposed to physiological saline in the trachea) in the normal group (6 mice), and pulmonary fibrosis model mice exposed to bleomycin in the trachea divided into 4 groups (control, compound A 50 mg/kg/day, losartan 50 mg/kg/day, and pirfenidone 400 mg/kg/day; 10 mice in each group). From the 7th day after exposure to bleomycin, a 0.5% aqueous solution of methylcellulose was orally administered (administered twice a day) to the normal group and the control group, the sample solution of compound A prepared above was orally administered (administered once a day) to the compound A administration group, the sample solution of losartan prepared above was orally administered (administered once a day) to the losartan administration group, and the sample solution of pirfenidone prepared above was orally administered (administered twice a day) to the pirfenidone administration group, respectively. Twenty-one days after the bleomycin exposure, the mice were anesthetized and whole lung tissues were extracted.

[Measurement Method]

The amount of hydroxyproline in the mice was measured by the same method as in Example 1.

[Measurement Result]

FIG. 3 shows the amount (μg) of hydroxyproline in the lungs of each group. In the figure, *** indicates a significant difference (t test) of p<0.001 as compared to the normal group. In addition, † indicates a significant difference (Dunnett test) of p<0.05 as compared to the control group. As is clear from FIG. 3, a significant increase in hydroxyproline in the lung was observed in the control group by intratracheal exposure to bleomycin. On the other hand, a significant decrease in hydroxyproline in the lung was observed in the compound A administration group as compared to the control group. Further, regarding the amount of hydroxyproline in the lung, no significant difference was observed between the losartan administration group and the pirfenidone administration group as compared to the control group.

A suppression effect of compound A on hydroxyproline accumulation in the lung was observed in the pulmonary fibrosis model animal induced by intratracheal exposure to bleomycin.

As described above, according to Examples 1 and 2, since administration of compound A of the present invention suppressed an increase in lung weight of the model animal of pulmonary fibrosis and further suppressed an increase in the amount of hydroxyproline in the lung, the compound A of the present invention was found to be useful in the prevention and treatment of pulmonary fibrosis related disorders such as idiopathic pulmonary fibrosis.

### INDUSTRIAL APPLICABILITY

The compound A of the present invention has industrial applicability because it is useful for preventing and/or treating pulmonary fibrosis related disorders.

1. A pharmaceutical composition comprising 3-[2-(5-{[1-(5-ethoxypyrimidin-2-yl)-2-isopropyl-6-oxo-4-propyl-1,6-dihydropyrimidin-5-yl]methyl}pyridin-2-yl)phenyl]-1,2,4-oxadiazol-5(4H)-one, a salt, a solvate or a solvate of the salt thereof for preventing and/or treating a pulmonary fibrosis related disorder.

2. The pharmaceutical composition according to claim 1, wherein the pulmonary fibrosis related disorder is a disease selected from the group consisting of idiopathic interstitial pneumonia, diffuse interstitial pneumonia, post-inflammatory pulmonary fibrosis, usual interstitial pneumonia, pulmonary fibrosis, diffuse alveolar damage, autoimmune disease and interstitial pneumonia caused by the autoimmune disease, pneumoconiosis, chronic hypersensitivity pneumonia and interstitial pneumonia caused by the chronic hypersensitivity pneumonia, drug-induced pneumonia and interstitial pneumonia caused by the drug-induced pneumonia, viral infectious disease and interstitial pneumonia caused by the viral infectious disease, radiation pneumonitis and interstitial pneumonia caused by the radiation pneumonitis, sarcoidosis and interstitial pneumonia caused by the sarcoidosis, pulmonary involvements associated with collagen vascular disease and interstitial pneumonia caused by the disease, and systemic scleroderma and interstitial pneumonia caused by the systemic scleroderma.

3. The pharmaceutical composition according to claim 1, wherein the pulmonary fibrosis related disorder is a disease selected from the group consisting of usual interstitial pneumonia, interstitial pneumonia caused by autoimmune disease, interstitial pneumonia caused by drug-induced pneumonia, interstitial pneumonia caused by viral infectious disease, interstitial pneumonia caused by radiation pneumonitis, interstitial pneumonia caused by sarcoidosis, interstitial pneumonia caused by pulmonary involvements associated with collagen vascular disease, interstitial pneumonia caused by systemic scleroderma, and idiopathic interstitial pneumonia.

4. The pharmaceutical composition according to claim 2 or 3, wherein the idiopathic interstitial pneumonia is a disease selected from the group consisting of idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia.

5. The pharmaceutical composition according to claim 1, wherein the pulmonary fibrosis related disorder is a disease selected from the group consisting of idiopathic pulmonary fibrosis, non-specific interstitial pneumonia, cryptogenic organizing pneumonia, desquamative interstitial pneumonia, respiratory bronchiolitis-associated interstitial lung disease, acute interstitial pneumonia, and lymphocytic interstitial pneumonia.

6. The pharmaceutical composition according to claim 1, wherein the pulmonary fibrosis related disorder is idiopathic pulmonary fibrosis.
